(19) United States
(12) Patent Application Publication
(10) Pub. No.: US 2023/0317228 A1
(43) Pub. Date: Oct. 5, 2023

(54) PATIENT CARE MANAGEMENT

(71) Applicant: Hill-Rom Services, Inc., Batesville, IN (US)

(72) Inventors: Patrice Etchison, Cary, NC (US); Craig M. Meyerson, Syracuse, NY (US); David Lance Ribble, Indianapolis, IN (US); Daniel Shirley, Raleigh, NC (US); Carlos Andres Suarez, Syracuse, NY (US); Gene J. Wolfe, Pittsford, NY (US)

(21) Appl. No.: 18/189,398

(22) Filed: Mar. 24, 2023

Related U.S. Application Data
(60) Provisional application No. 63/362,250, filed on Mar. 31, 2022.

(51) Int. Cl. G16H 20/00

(52) U.S. Cl. CPC G16H 20/00; G16H 15/00

(57) ABSTRACT

An example unified application can leverage the insights gained about the patient to provide a range of customized services that aid in the patient's recovery throughout the recovery process. The disclosed system and methods provide a continuum of care for the patient, including services that aid in the patient's recovery and management of a medical condition outside the hospital environment, pre-hospitalization and post-hospitalization.

### BACKGROUND

Clinical care is only one step along the path to helping patients achieve their optimal state of health. Patients must be engaged and activated to participate in their own care in order for clinical services to be truly effective. Patient activation has been shown to cut costs and improve care outcomes. When patients effectively manage their own health, they are more likely to obtain overall wellness.

### SUMMARY

In general terms, the present disclosure relates to a unified patient care system that increases patient engagement and efficiencies in patient care. Various aspects are described in this disclosure, which include, but are not limited to, the following aspects.

In one aspect, an example method for providing patient care is disclosed. The method comprises: delivering pre-hospital information to a patient in a graphical user interface, the pre-hospital information including information about possible treatments for the patient and a pre-operation virtual consult with a caregiver; delivering post-hospital information to the patient in the graphical user interface, the post-hospital information including information about medications and rehabilitation for the patient.

In another aspect, an example method for providing patient care outside of a healthcare facility is disclosed. The method comprises: identifying needs of a patient located outside of the healthcare facility; routing one or more of the needs to a caregiver of the patient; providing a dashboard for the patient, the dashboard including: capturing patient information from the patient and the caregiver; and accessing the patient information for the caregiver.

In yet another aspect, an example method of gamification of a patient care is disclosed. The method comprises: identifying a health condition of a patient; identifying an electronic game that aids in improving the health condition; and providing the game on a user electronic computing device of the patient, wherein the patient engages with the electronic game through the user electronic computing device.

### DESCRIPTION OF THE FIGURES

The following drawing figures, which form a part of this application, are illustrative of the described technology and are not meant to limit the scope of the disclosure in any manner.

FIG. 1 illustrates an example configuration of a patient care management system.

FIG. 2 illustrates an example configuration of a patient care management engine of the system of FIG. 1.

FIG. 3 illustrates an example patient care management user interface of the system of FIG. 1.

FIG. 4 illustrates an example care team summary user interface of the system of FIG. 1.

FIG. 5 illustrates an example resources user interface of the system of FIG. 1.

FIG. 6 illustrates an example search articles interface of the system of FIG. 1.

FIG. 7 illustrates an example user request user interface of the system of FIG. 1.

FIG. 8 illustrates an example transportation request user interface of the system of FIG. 1.

FIG. 9 illustrates an example log patient data user interface of the system of FIG. 1.

FIG. 10 illustrates an example games user interface of the system of FIG. 1.

FIG. 11 illustrates an example interactive game user interface of the system of FIG. 1.

FIG. 12 illustrates an example game statistics user interface of the system of FIG. 1.

FIG. 13 illustrates an example of a device that can be used to implement aspects of the patient care management system of FIG. 1.

### DETAILED DESCRIPTION

The present disclosure relates to a patient care management system that integrates disparate patient related data in a unified application to increase efficiencies of patient care. In general terms, the disclosed system facilitates patient care before, during and after a clinical visit or hospitalization by improving communications between patients and their care team and providing solutions to improve engagement of patients with their care. Many different examples are provided below.

Successful patient outcomes can be directly correlated to the level of engagement and compliance of a patient with the care provided. Augmenting the level of engagement is of significant interest in the application of care. A mechanism is disclosed that can do one or more of the following: 1) keep all members of the patient's care team on the same page by allowing for open and continuous communication between the members; 2) continually support the needs of the patient, including pre- and post-hospitalization and/or clinical visits; 3) provide continual access to information regarding the patient's care plan; 4) connect the patient with resources and services that will aid in the patient's recovery; 5) deliver an engagement experience; and 6) monitor and quantize that engagement is of high value.

The present disclosure is directed to a unified application that leverages the insights gained about the patient to provide a range of customized services that aid in the patient's recovery throughout the recovery process.

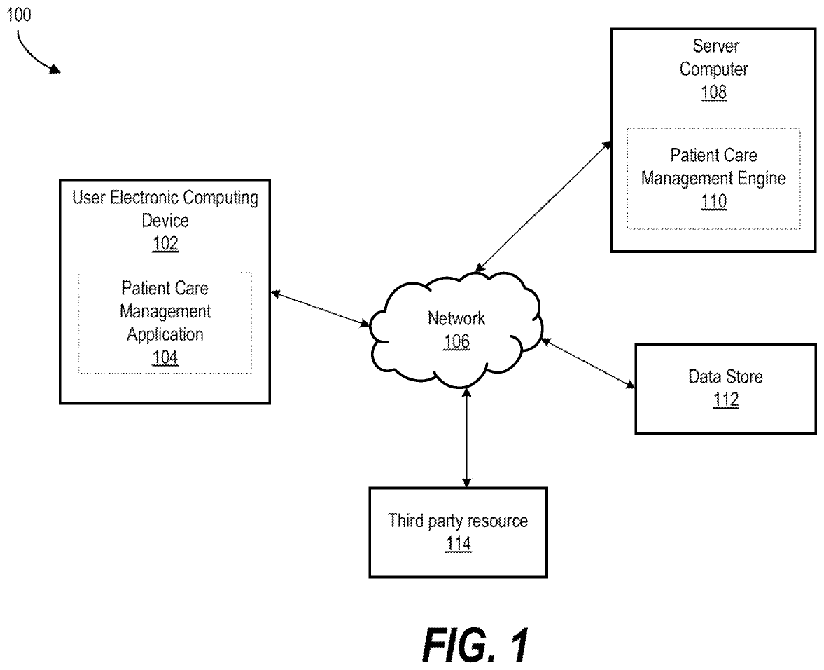

FIG. 1 illustrates an example configuration of a patient care management system 100. The patient care management system 100 may include one or more user electronic computing devices 102, a network 106, a server computer 108, one or more datastores 112, and one or more third party resources 114. In some examples, the server computer 108 may include a patient care management engine 110.

In some examples, user electronic computing device 102 is an electronic computing device of the user. In some examples, the electronic computing device can be a desktop computer, a laptop computer, virtual reality user device, a mobile electronic computing device such as a smartphone or a tablet computer. The electronic computing device permits the user to access the server computer 108 over a network 106. Although a single user electronic computing device 102 is shown, the patient care management system 100 allows hundreds, thousands, or more computing devices to connect to the server computer 108.

For example, a patient care management application 104 may be installed on the user electronic computing device 102. In some examples, the patient care management application 104 may generate and display a user interface on a display screen associated with the user electronic computing device 102 that allows the user to access patient care related features of the patient care management engine 110. In other examples, the user interface may be generated by the patient care management engine 110 and send to the patient care management application 104 for display on the display screen associated with the patient care management engine 110.

In some examples, the user may include a patient, or a caregiver. Other types of users are also possible. For example, a caregiver to the patient may include an informal caregiver such as a family member or friend of the patient, a professional at-home caregiver, a staff member within a rehabilitation facility or anyone else that cares for the health of the patient outside a clinical setting, a healthcare provider such as a physician, nurse, surgeon, therapist, specialist or any other medical professional or administrative staff associated with a medical facility from which the patient may seek care.

In some examples, the network 106 is a computer network, such as the Internet. The user associated with the user electronic computing device 102 can access the patient care management engine 110 in the server computer 108 via the network 106.

In some examples, the server computer 108 is a server computer of healthcare facility, such as a hospital or clinic, or a server computer of third-party patient information management company. Although a single server is shown, in reality, the server computer 108 can be implemented with multiple computing devices, such as a server farm or through cloud computing. Many other configurations are possible.

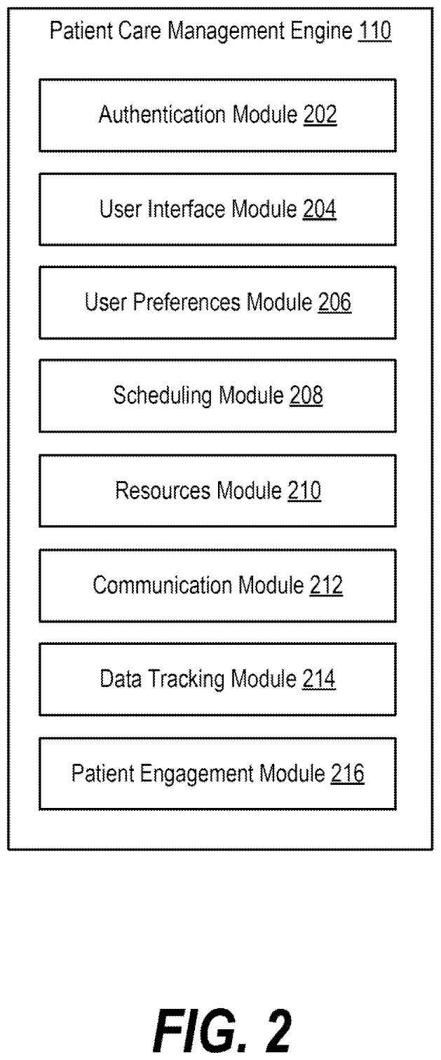

In one example, the patient care management engine 110 is configured to receive patient related information and requests from the user electronic computing device 102 and manages the received information and requests appropriately. The implementation of the patient care management engine 110 is described in further detail in relation to FIG. 2.

The example datastore 112 may include one or more electronic databases that can store data related to the user and/or patient care management engine 110. The datastore 112 may be maintained by the same entity that maintains the server computer 108 or one or more external enterprises associated with the entity that maintains the server computer 108. The datastore 112 can be accessed by server computer 108 to retrieve relevant data associated with the user and the patient care management engine 110.

The patient care management engine may interface with a third-party resource 114 in response to a request from the user. For example, when a user requests assistance related to a resource or service provided by a third-party resource 114, the patient care management engine 110 may submit the request to a third-party resource 114 through an Application Programming Interface (API). The third-party resource 114 may in turn receive the request, process the request and provide a response to the request directly to the user electronic computing device 102 or to the patient care management engine 110, which in turn sends the response to the user electronic computing device 102. Although a single third-party resource 114 is shown, in reality, the patient care management engine 110 may interface with multiple third-party resources. Some third-party resources may include applications for entities directed at food delivery, grocery delivery, medication refills and delivery, cleaning services, medical equipment, transportation services. Other types of third-party resources are also possible.

FIG. 2 illustrates an example configuration of the patient care management engine 110. The patient care management engine 110 includes an authentication module 202, a user interface module 204, a user preferences module 206, a resources module 210, a scheduling module 208, a communications module 212, a data tracking module 214 and a patient engagement module 216. More of fewer modules are also possible.

The patient care management engine 110 may be configured to allow a user, including the patient or the patient's caregiver, to engage with the patient's care by accessing medical information related to the patient, communicating with the members of the patient's care team, retrieving educational information related to the patient's condition, possible treatment options, scheduling appointments, logging patient health related information, making requests that aid the patient during recovery, and providing interactive activities that engage the patient and aid in recovery. The patient care management engine 110 may also include other types of features.

The authentication module 202 may be configured to authenticate the user. For example, the authentication module 202 may generate an authentication user interface that may be sent to the user electronic computing device 102 for display on a display screen associated with the user electronic computing device 102. The authentication user interface may request the user of the user electronic computing device 102 to provide authentication information. Authentication information may include a username/password, biometrics, FOB/scanner, etc. Authentication of the user may also be performed by using real time location systems (RTLS) to locate the user and/or a user device associated with the user and automatically authenticating the user based on the user's location. Upon receiving authentication information, the user electronic computing device 102 may send the authentication information to the authentication module 202 for confirmation. The authentication module 202 may compare the received authentication information with the authentication information stored in the datastore 112 for confirmation of the identity of the user.

Once the identity of the user is confirmed, the user interface module 204 of the patient care management engine 110 may generate a patient care management user interface for display on the user electronic computing device 102. The patient care management user interface may be customized based on the identity of the user. The patient care management user interface may then be sent to the patient care management application 104 for display on the user electronic computing device 102. Alternatively, the user interface module 204 of the patient care management engine 110 may send the appropriate data to the patient care management application 104 and the patient care management application 104 may generate the patient care management user interface.

The information and features displayed on the patient care management user interface may vary based on the identity of the user. For example, the patient care management user interface of a user that is a patient may differ from the patient care management user interface screen for a user that is a caregiver. For example, a patient may have access to the complete medical history of the patient while a caregiver may simply have access to a current medication list and not the entire medical history of the patient.

The user interface module 204 may be configured to interface with the user preferences module 206, the resources module 210, the scheduling module 208, the communications module 212, the data tracking module 214 and/or the patient engagement module 216 in order to generate user interface displays that display the relevant information based on the identity of the user, the selections made by the user and the user's preferences.

The user preferences module 206 may be configured to receive and store preferences of the user. The user preferences module 206 may store different types of data depending on the identity of the user. In one example, for a caregiver that is helping a patient outside of a clinical setting, the user preferences module 206 may store data including, the name of the user, the name of the patient that the user is caring for, the patient's account or identification number, the relationship of the user to the patient, user setting preferences. Other types of data regarding the caregiver user may also be collected and stored. In another example, for a user that is a patient, the user preferences module 206 may collect and store data including, name, age, residential address, medical history information, dietary preferences, allergen information, dietary restrictions, caregiver related preferences including designation of primary or secondary caregiver status, emergency contact information, which caregiver to route request responses to, socio economic status, preferences regarding nutrition, pharmacy, grocery, transportation, medical equipment and cleaning services. Other types of data regarding the patient user are also possible.

In some examples, the user preferences module 206 may also be configured to receive and store user preferences related to access rights. For example, a user that is a patient may be able to control access rights for different caregivers, including the types of information that each caregiver can access. For example, the user, through the patient care management application 104 at the user electronic computing device 102 may be able to designate that user A is a primary caregiver and may have access to the patient's current medical communications, notes and medications and designate that user B is a secondary caregiver and may have access to the patient's requests and responses to requests, but not to the patient's current medical notes, communications, or medications.

The scheduling module 208 may be configured to provide a schedule of past and future medical appointments and healthcare events. For example, pre-hospitalization or before a clinical visit, the scheduling module 208 may be configured to list out the list of events that are scheduled for the user during their visit or hospitalization. The scheduling module 208 may also be configured to list the discharge schedule and post discharge follow-up appointment schedule. The scheduling module 208 may also auto-schedule follow up appointments based on an analysis of the user's condition and discharge summary and instructions. For example, when a patient is discharged after delivering a baby, the scheduling module 208 may auto-schedule a post-partum appointment with the user's physician for six-weeks after the discharge date.

The scheduling module 208 may also be configured to allow the user to schedule future appointments, healthcare visits and consultations. For example, the user, through the patient care management application 104 on the user electronic computing device 102 may input dates, times, name of a healthcare provider in order to schedule an appointment or consultation with the healthcare provider.

In some examples, the user may use the scheduling module 208 to schedule a virtual consult with a caregiver prior to a hospitalization, clinical visit or operation. The scheduling module 208 may interface with the communications module 212, described in greater detail below, to facilitate the virtual consult. For example, the user may provide be able to access the calendar of a caregiver and setup a virtual consult in order to discuss questions regarding the upcoming appointment or procedure.

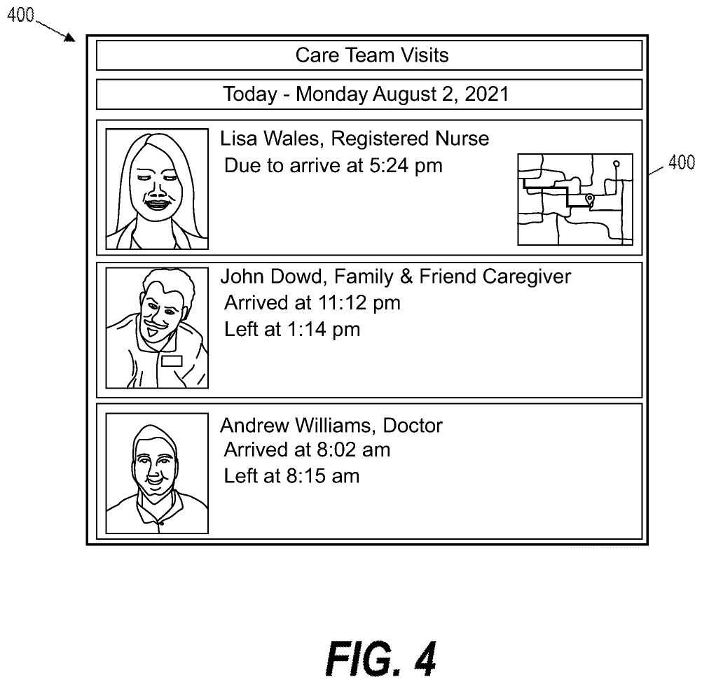

In some examples, when the user receives assistance from one or more caregivers, the scheduling module 208 may also provide a schedule associated with the caregivers. For example, as detailed further in relation to FIG. 4, the scheduling module 208 may cause the patient care management application 104 to list a schedule of past and upcoming care team visits.

The resources module 210 is configured to provide the user with resources that may be helpful to the user in managing the user's health. For example, the resources module 210 may provide the user with educational materials related the user's medical condition(s) and possible treatment options. In addition, when the user is scheduled for a procedure, hospitalization or clinical visit, the resources module 210 may make all materials related to the procedure, hospitalization or clinical visit available for the user to review before the visit. The resources module 210 may also make all materials related to discharge or post-visit care available for the user to review. For example, post-visit care may include a list of medications, instructions regarding the medications, rehabilitation exercise information, restrictions regarding nutrition, fluid intake and physical activity, follow-up appointment schedule, etc.

The resources module 210 may receive links or uploaded educational documents or materials from a caregiver associated with the patient or may automatically retrieve educational materials that may be related to the patient's condition and treatment from one or more datastores 112 based on the patient's upcoming appointments, current medications, medical history, etc.

In some examples, the user may be required to review educational materials before and/or after the clinical or hospital visit. In such cases, the resources module 210 may interface with the data tracking module 214 to track the user's progress as the user scrolls through documents. The tracking of data is further described in relation to the data tracking module 214 below.

In addition to educational documents and materials, the resources module 210 may also provide the user with a list of experts specializing in the patient's condition. For example, the resources module 210 may retrieve healthcare providers that have expertise with the user's health condition within a predetermined distance from the user's residential location and send the list of experts to the user electronic computing device 102 via the user interface module 204, where the list of experts may be displayed on a user electronic computing device 102 for user review. In addition to displaying a list of experts, the resources module 210 may also recommend particular healthcare providers or experts based on the user's preferences for providers. In some examples, a machine learning algorithm may be used to assist in selecting a healthcare provider based on the user's preferences and history with healthcare providers.

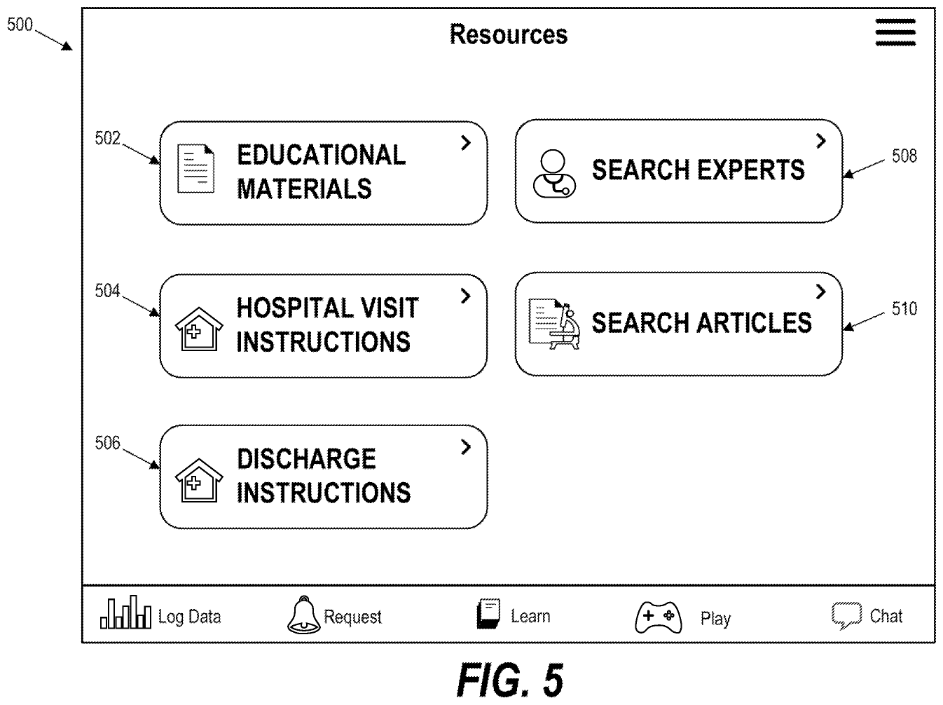

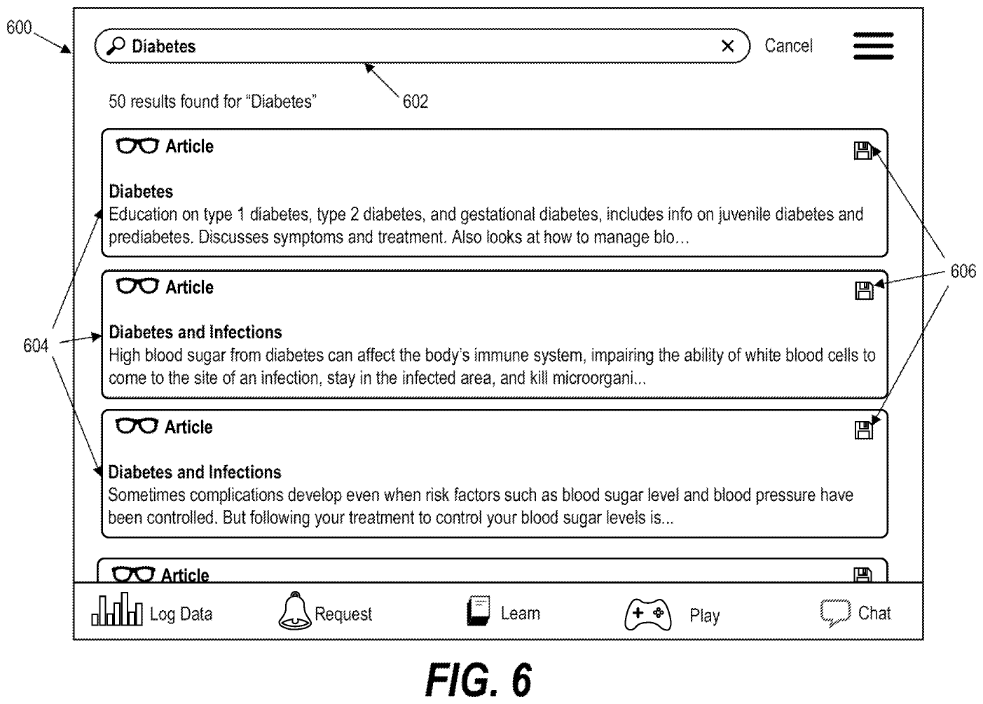

The resources module 210 may also be configured to include a search feature that allows the user to search for specific experts, articles, publications, etc. The search feature, as well as other features configured by the resources module 210 are further described in relation to FIGS. 5 and 6.

The communications module 212 may be configured to facilitate communications between users. For example, the communications module 212 is configured to facilitate communications between a patient and a caregiver for the patient, including a healthcare provider for the patient. For example, the communications module 212 may provide a messaging feature that allows a patient to send and receive messages to and from their healthcare provider(s) and/or other caregivers.

In another example, the communications module 212 may be configured to interface with the scheduling module 208 to setup a pre-hospitalization, or pre-operation virtual consult with a caregiver. In some examples, the communications module 212 may be configured to send, receive and process streaming audio and video files by using a camera and microphone associated with the user electronic computing device 102. In other examples, the communications module 212 may be configured to interface a third-party communications application such as Microsoft Teams, Zoom or Skype in order to facilitate the virtual consult.

In yet another example, the communications module 212 may also be configured to receive questions or queries from the user and route the question to the correct end user. In some examples, the communications module 212 may facilitate communications between the patient and a specific caregiver. In other examples, the communications module 212 may include a message board feature that facilitates communication among multiple users including the patient, an entire team of informal and formal caregivers such as family members and healthcare providers. For example, a healthcare provider may post that the patient cannot receive food after 9 PM due to planned lab work the following day and everyone on the patient's team, including the patient, and all of the patient's caregivers may be able to view the message simultaneously.

The communications module 212 may also be configured to integrate a chatbot feature that may use an artificial intelligence algorithm or software application that is trained to automatically answer questions from a user without human intervention. For example, the patient care management application 104 may display a chat feature where the user may enter a question by typing in the question or using voice command. The question may then be sent to the patient care management engine 110 on the server computer 108 via the network 106, where the communications module 212 may use the integrated artificial intelligence algorithm or software application to generate a response to the question and send the response to the patient care management application 104 for display on the user electronic computing device 102.

In some examples, the communications module 212 may be configured to receive a user request and interface with one or more third party resources 114 to fulfill or respond to the request. For example, a user who is a patient that is currently recovering from a foot surgery may make a request for assistance with grocery shopping, food delivery, medication delivery, transportation to and from an appointment or other types of activities.

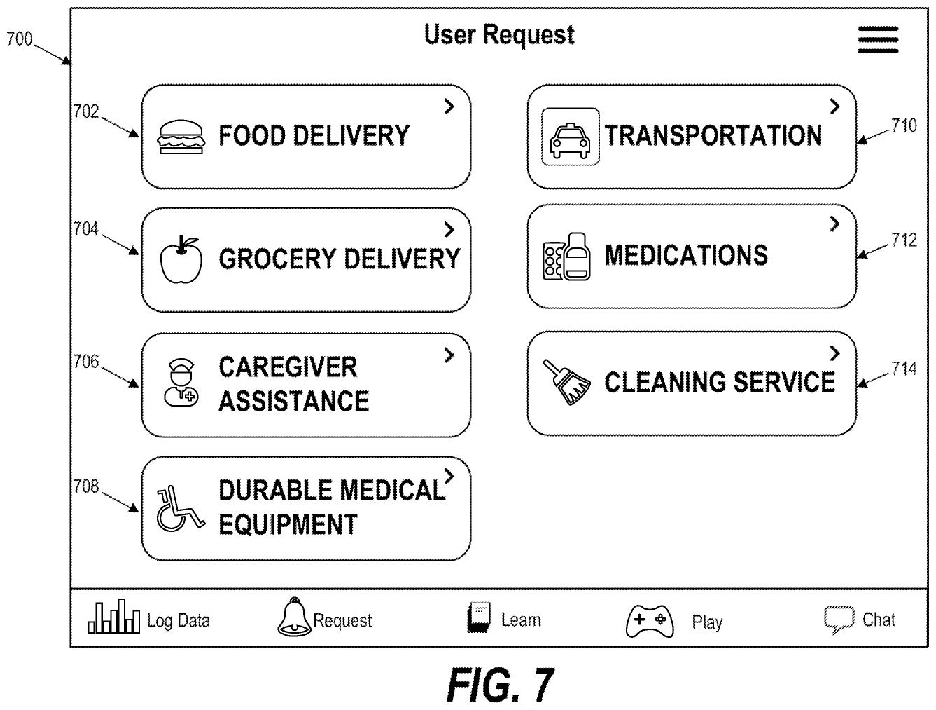

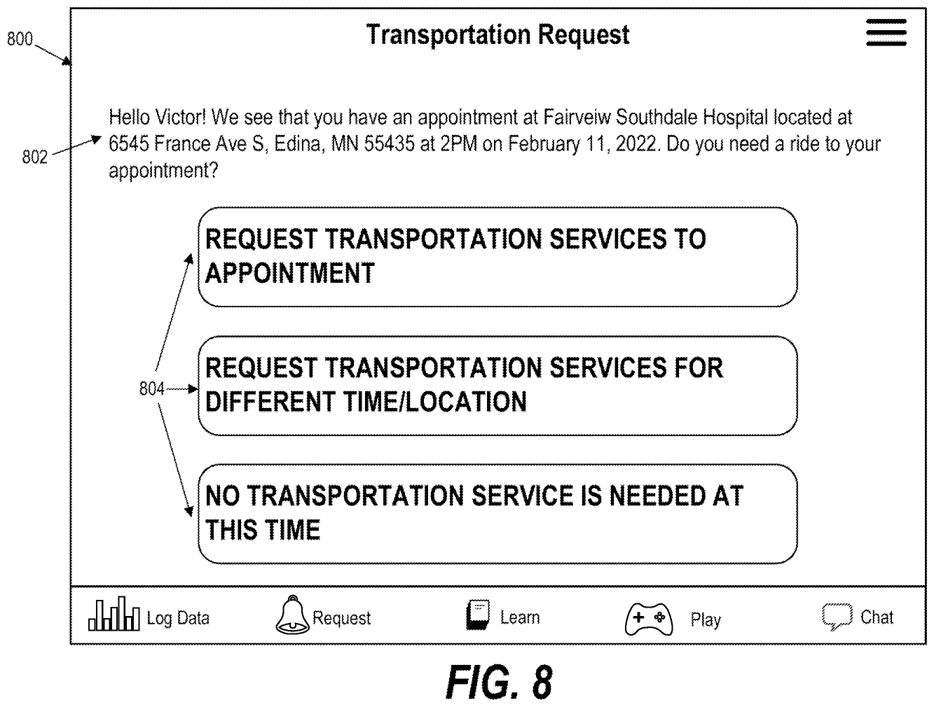

For example, the patient care management engine 110 may identify the user has an appointment with their primary care provider at 2 PM the next day and automatically offer the user to assist with scheduling transportation to the location at the appropriate time. Alternatively, the user may request transportation to the user's next appointment that is schedule for 2 PM the next day using the patient care management application 104. The patient care management engine 110 may receive and process the request based on the user's preferences for services. For example, the user may have previously indicated that they prefer Uber for transportation services. Thus, the patient care management engine 110 may interface with the API associated with an Uber application and schedule a ride for the user at 2 PM the next day from the user's preferred location to the location of the clinic where the appointment is scheduled. The feature related to the user requests is described in greater detail in relation to FIGS. 7-8 below.

In some examples, the communications module 212 may interface with a commercially available voice-assistance technology such as Amazon Alexa or Google Home to receive and process voice commands. The voice assistance technology may be used to receive and process user requests for one or more services such as nurse calls, including third-party services. The voice assistance technology may also be used to gather user data such as contextual data, location data, ambulation data and pain mitigation data. The gathering and tracking of user data are further described in detail in relation to the data tracking module 214 described below.

The data tracking module 214 may be configured to track user data. The type of data that is tracked may differ between different types of users. For example, in cases where the user is a patient, the data tracking module 214 may track patient related data such as the user's nutrition, fluid intake, the dosage and timing of medication intake, the user's activity history, the user's vitals, the user's medical condition. Other types of medical data related to the user may also be tracked. In some examples, the patient data may be received from a user that inputs the data into the patient care management application 104. In other examples, the patient data may be directly received from one or more smart devices such as the user's smart watch, a Bluetooth or wireless connected scale, blood pressure monitor, glucometer or other medical devices that may directly communicate with the server computer 108 through a network 106.

In other examples, the data tracking module 214 may track the user's compliance with pre-hospitalization or pre-operation requirements by tracking whether the user's nutrition and fluid intake meet the requirements outlined by the user's healthcare provider. The data tracking module 214 may also track the user's compliance with hospitalization or clinical care procedures by tracking whether the user has spent a pre-determined amount of time, scrolled through, accessed or otherwise reviewed all or portions of the educational materials, admission forms, consent forms and other documents related to the user's upcoming hospitalization, procedure or operation.

In yet other examples, the data tracking module 214 may also track data related to user's engagement level with their care by tracking the user's engagement with the patient care management application in general, as well as the user's engagement with the one or more games configured by the patient engagement module 216. The patient engagement module 216 is described in greater detail below.

In some examples, user data related to a patient may be received from the patient itself or from the patient's caregiver, such as an informal caregiver that provides care for the patient outside the clinical setting. The data tracking module 214 may store the received user data in a datastore 112 or process and/or send the data to a healthcare provider associated with the user. In some examples, when compliance of the user to a pre-hospitalization requirement is required to proceed with the hospitalization, the data tracking module 214 may track whether the user has been compliant and may send a compliance report indicating whether the user passed or failed to comply with the requirements to the appropriate healthcare provider or hospital administrator prior to the hospitalization.

In other examples, the data tracking module 214 may receive user data regarding one or more of patient's nutrition, medication, fluid intake, vitals, exercise, activity level, progress in patient's condition, laboratory results and generate automatic reports and documents outlining the patient's condition and store the report in datastore 112 or send the report to the appropriate healthcare provider upon request or automatically on a periodic basis.

When the user is a caregiver associated with a patient, the data tracking module 214 may also receive data from the user regarding when the user arrived to care for the patient and when the user departed after caring for the patient. For example, the user may track arrival and departure times by using a badge reader, near field communication, RFID, barcode reader, or using GPS location of the user using the user's mobile device. The user may also simply log in their arrival and departure times by entering the times in the patient care management application 104.

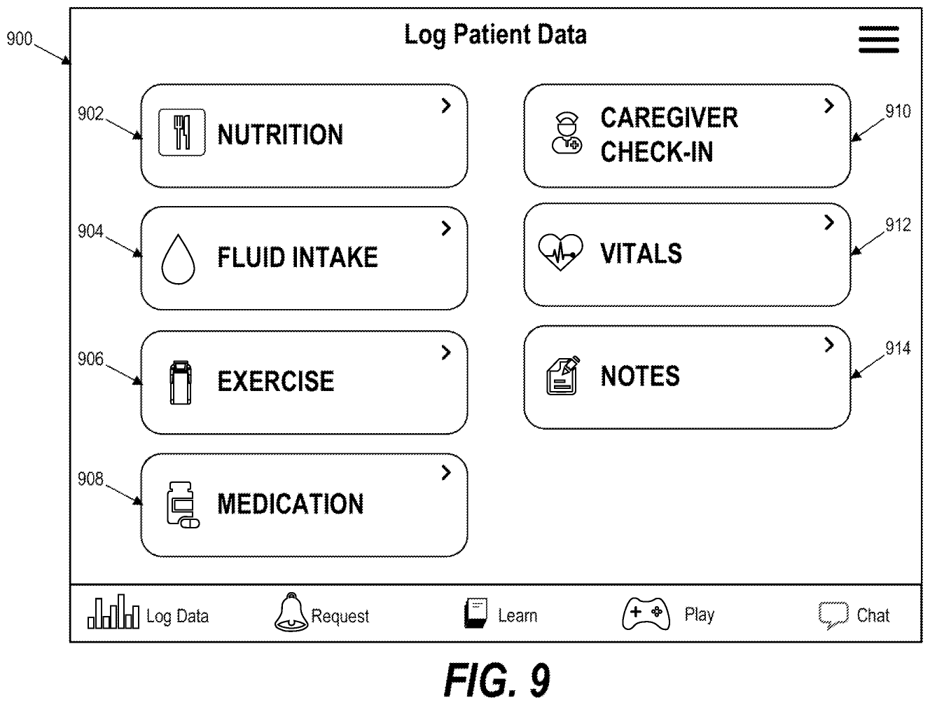

In some examples, the user, who is a caregiver, may also record all activities performed by the user during their time with the patient. For example, the user may record the food, fluids, medication consumed by the patient along with timestamps and notes. The data tracking module 214 may store the user data received from the user and/or may process the data to create reports or documents that may later be sent to the patient's healthcare provider. The features configured by the data tracking module 214 is further described in relation to FIG. 9.

The patient engagement module 216 may be configured to provide an interactive activity or game to benefit a user that is a patient by improving the health condition of the patient. For example, the patient engagement module 216 may provide one or more interactive activities or games that are specific to the user to improve the user's health condition. The patient engagement module 216 may track user engagement with the interactive activity or game and generate a score to indicate the user's progress with the interactive activity or game.

In some examples, the interactive activity or game may relate to improving the user's health condition. For example, a diabetic patient may be presented with their blood glucose score through the day as a car travelling along a bumpy road—with the goal to keep the car as stable as possible. A COPD patient with a spirometer may be told to blow a graphic of a sailboat across a river, wherein each exhale pushes the boat virtually further down the water. Other types of interactive activities or games to improve the user's health condition are also possible.

For example, the patient engagement module 216 may analyze the user's health history and current health condition to automatically provide one or more interactive activities or games for the user to choose from. The patient engagement module 216 may access the one or more datastores 112 to retrieve relevant interactive activities or games from a plurality of stored interactive activities or games. The one or more relevant interactive activities or games may then be sent to and presented on the patient care management application for the user to select from. The user's progress through the selected interactive activities or games may be tracked. For example, the number of levels in the activity or game completed by the user, the user's current level of the activity or game, total points score so far, the user's level of engagement with the game may be tracked and displayed for the user to review.

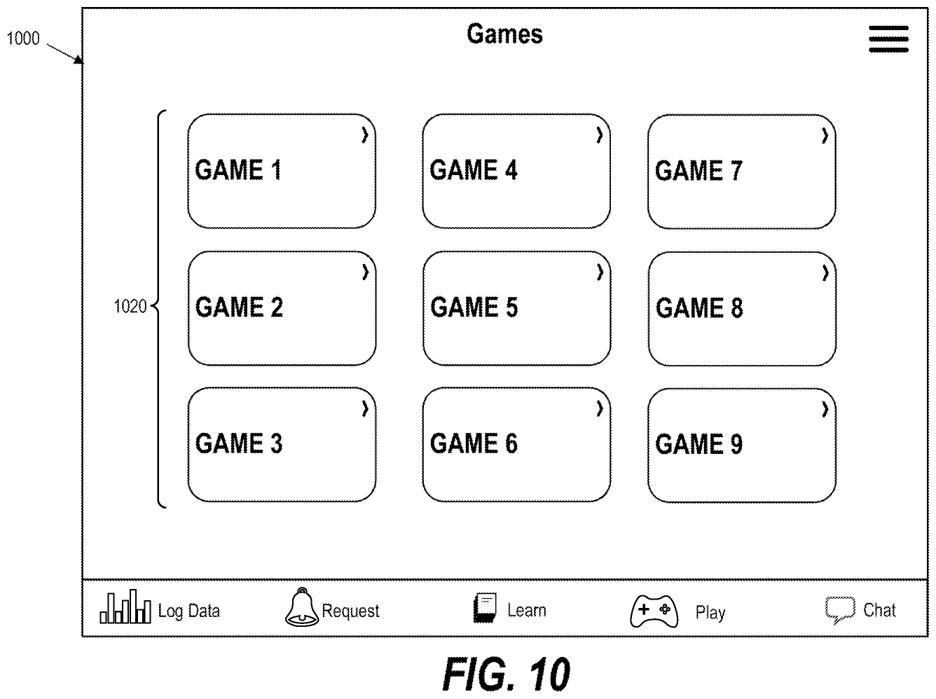

For example, the patient engagement module 216 may be configured to provide a list of one or more rewards on the patient care management application 104. A user may be able to select one or more rewards from the list of rewards based on the number of points accumulated by the user by completing all or portions of the interactive activities or games. The features related to the patient engagement module 216 are further described in relation to FIGS. 10-12 below.

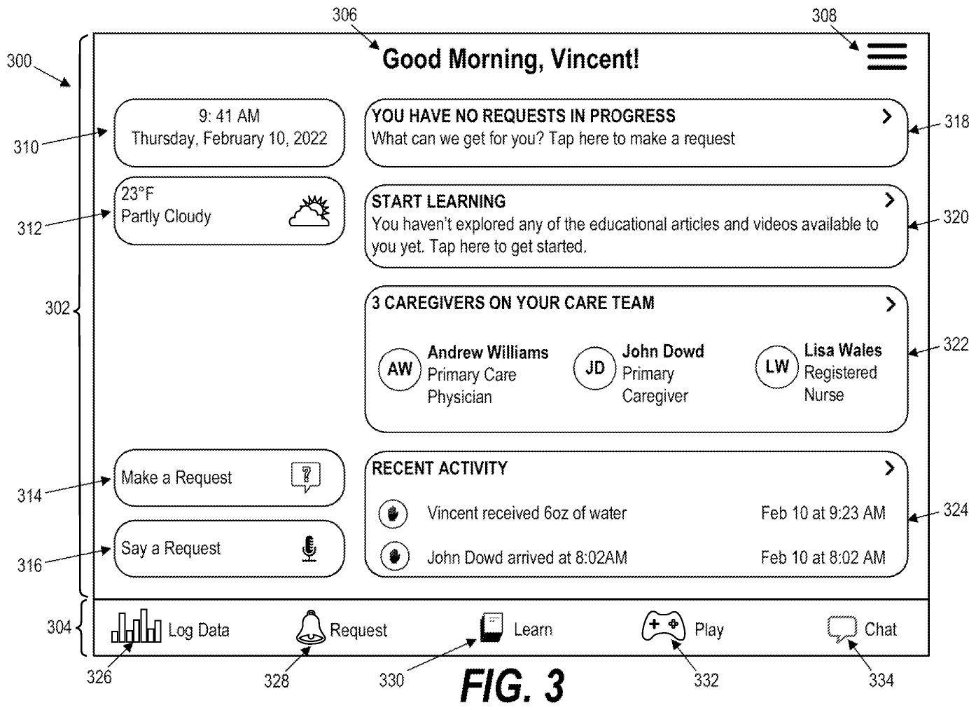

FIG. 3 illustrates an example user interface representing a patient care management user interface 300. The patient care management application 104, upon being executed on the user electronic computing device 102, may be configured to receive instructions from the patient care management engine 110 to display the patient care management user interface 300 on a display screen of the user electronic computing device 102. In some examples, the patient care management user interface 300 may be a dashboard that includes a plurality of features related to patient care that is accessible to the user.

In some examples, the features and content included with the patient care management user interface 300 may be generated by the user interface module 204 of the patient care management engine 110 and sent to the patient care management application 104 for display on the user electronic computing device 102 as the patient care management user interface 300. In other examples, the patient care management application 104 itself may generate the patient care management user interface based on information received from the patient care management engine 110.

Although in FIGS. 3-12 illustrate example user interface for a mobile electronic device, the patient care management application may be installed and executed in any electronic computing device. As such the layout and visual representation of the features described in relation to FIGS. 3-12 may be different than illustrated in FIGS. 3-12.

The patient care management user interface 300 may in include a main display area 302 and a tab bar area 304. The main display area 302 may display content related to the selected page. In the case of the patient care management user interface 300, the main display area 302 may include a welcome note 306, a list menu 308, a date/time display 310, a local weather display 312, one or more options to make user requests and one or more selectable options related to the patient care management features.

For example, the welcome note 306 may include a greeting for the user to indicate that the user is now logged in and the patient care management user interface 300 is personalized according to the user's preferences. In the present example patient care management user interface 300, the welcome note 306 includes the message “Good Morning, Vincent!.” Other types of welcome notes are also possible based on the user's preferences.

For example, the list menu 308, when selected, contains a group of navigation links to different aspects of the patient care management application 104. For example, the list menu 308 may include selectable navigation links to display pages that focus on different features associated with the patient care management application 104, including, but not limited to: user authentication, user preferences, user schedule, resources, communications, user requests, log user data, and interactive activity/games. Selecting any of the navigation links included with the list menu 308 would result in the patient care management application 104 updating the user interface screen to display the relevant user interface display. FIGS. 4-12 include some example user interface displays illustrating some of the features of the patient care management application 104.

The date/time display 310 displays the local date and time of the user. The local weather display 312 displays the local weather at the location of the user. The date/time display 310 and local weather display 312 may be displayed to personalize the patient care management application 104 for the user. In some examples, more, less or different types of display of information personal to the user may also be included in addition to the date/time display 310 and the local weather display 312.

The patient care management user interface 300 may also include one or more options to make user requests. For example, the patient care management user interface 300 may include a selectable “make a request” option 314. Upon selecting the “make a request” option 314, the patient care management application 104 may navigate to a user request user interface 700 display that includes one or more options that allow the user to make a request related to services that would assist the user due to the user's health condition. The user request user interface 700 may be described in further detail in relation to FIG. 7.

The one or more options to make user requests may also include a “say a request” option 316. For example, the “say a request” option 316 may be configured to receive voice commands regarding user requests from the user. The “say a request” option 316 may be integrated to a voice assistance application such as Amazon Alexa or Google Home such that the voice command from the user may be received and sent to the communications module 212 of the patient care management engine 110 for further processing without the user having to select the “make a request” option 314 and select options on the user electronic computing device 102.

The patient care management user interface 300 may also include one or more selectable options related to the one or more patient care management features. Each of the one or more selectable options may be associated with a particular feature of the patient care management application 104. Selecting a selectable option from the one or more selectable options allows the user to navigate to a user interface focused on the feature associated with the selected selectable option.

The one or more selectable options that are displayed on the main display area 302 may be customizable by the user. For example, the user, using the user preferences option from the list menu 308, may select the selectable options to be included in the main display area 302. The selectable options, in addition to providing a method of navigating to a user interface focused on the feature associated with the selectable options, may also provide a brief personalized summary of the feature itself.

In the example from FIG. 3, the one or more selectable options include a user requests selectable option 318, a learning selectable option 320, a care team listing selectable option 322, and a recent activity selectable option 324.

The user requests selectable option 318 may be configured to include a brief personalized summary of the status of the user requests. For example, in the present example from FIG. 3, the user requests selectable option 318 includes a summary that the user does not have any requests in progress. Selecting the user requests selectable option 318 may navigate the user to a user request user interface 700, such as the one described in further detail in relation to FIG. 7 below.

The learning selectable option 320 may be configured to include a brief personalized summary of the status of the learning. For example, in the present example from FIG. 3, the learning selectable option 320 includes a summary that the user has not started exploring any of the educational articles and videos available to the user. Alternatively, if the user has started viewing a required educational video, but has not completed the playback yet, the summary may state that the user needs to complete viewing the video before the user's next appointment. Selecting the learning selectable option 320 may navigate the user to a resources user interface, such as the resources user interface 500 described in relation to FIG. 5 below.

The care team listing selectable option 322 may be configured to include a brief personalized summary of the status of the user's care team. For example, in the present example from FIG. 3, the care team listing selectable option 322 includes a summary of the name and title of each member of the team of care providers that care for the user, including a primary care physician, a primary caregiver and a registered nurse. Selecting the care team listing selectable option 322 may navigate the user to a user interface, similar to that illustrated in FIG. 4, that lists a summary of the care team and a schedule of arrival/departure times for the care team.

The recent activity selectable option 324 may be configured to include a listing of all recent user activity associated with the patient care management application 104. For example, in the present example from FIG. 3, the recent activity selectable option 324 includes a summary of the user's recent activity including the arrival of a caregiver and a log of the user's fluid consumption. Selecting the recent activity selectable option 324 may navigate the user to a user interface that lists all recent user activities according to the timeline of events, with the most recent activities listed at the top of the list.

In some examples, the tab bar area 304 may be configured to be located below the main display area 302 and include one or more selectable icons to assist the user in easily navigating to commonly used features of the patient care management application 104. The one or more selectable icons that are included within the tab bar area 304 may be customizable. For example, each user may, within the user preferences option of the list menu 308, select their most commonly accessed features of the patient care management application 104 to be added to the tab bar area 304.

In the present example, the tab bar area 304 includes a selectable log patient data icon 326 that provides a shortcut to the log patient data user interface 900 described further in relation to FIG. 9, a selectable user request icon 328 that provides a shortcut to the user request user interface 700, described further in relation to FIG. 7, a selectable learn icon 330 that provides a shortcut to the educational materials option of the resources user interface 500, described further in relation to FIG. 5, a selectable play icon 332 that provides a shortcut to the user's last played game or interactive activity from a user engagement user interface 1000, described further in relation to FIG. 10 and a selectable chat icon 334 that provides a shortcut to the chat feature that facilitates the user to send questions or queries to an automated chatbot for quick responses, as described in relation to the communications module 212.

FIG. 4 illustrates an example care team summary user interface 400. The example care team summary user interface 400 may be configured to receive instructions from the patient care management engine 110 to display a list of members on the user's care team, including a name and photograph of each care team member and a summary of arrival/departure times of the members of the care team. In some examples, where the user is a caregiver, the care team summary user interface 400 may include a list of other care team members associated with each patient the user cares for.

In the present example, the care team for a particular user includes three members including Lisa Wales, a registered nurse, John Dowd, a family and friend caregiver and Andrew Williams, a doctor. Each of the members of the care team are listed along with their last arrival and departure times. Alternatively, if the care team member has not arrived for the day, a listing of when the care team member is due to arrive may be listed instead. In the present example, Andrew Williams arrived at 8:02 AM and left at 8:15 am and John Dowd arrived at 11:12 am and left at 1:14 pm. Lisa Wales is indicated as being due to arrive at 5:24 pm.

In some examples, the care team summary user interface 400 may be generated by the patient care management engine 110 using data from the scheduling module 208 and the data tracking module 214 that track when each care team member's schedule as well as the arrival and departure times of the care team members using a one or more of badge readers, near field communication, RFID, barcode readers, and GPS location tracking.

For example, when a user who is a care team member has enabled GPS location tracking on their user electronic computing device 102, the data tracking module 214 may track the user's GPS location when the user is due to arrive to care for another user, who is a patient. For example, based on the data from the data tracking module 214, the care team summary user interface 400 may include a map 402 with a care team member's current location for any team members that are due to arrive shortly so the patient user may estimate when to expect the care team to arrive.

FIG. 5 illustrates an example resources user interface 500. The example resources user interface 500 may be configured to receive instructions from the patient care management engine 110 to provide selectable options to one or more resources for the user. For example, the selectable options to one or more resources may be customized to the needs of the user. In the present example, the example resources user interface 500 may include a educational materials option 502, a hospital visit instructions option 504, a discharge instructions option 506, a search experts option 508 and a search articles option 510. The list of selectable options may differ between users. For example, the resources user interface 500 of the patient care management application 104 for a user that was not hospitalized may not include the discharge instructions option 506.

For example, selecting the educational materials option 502 may display one or more documents, videos, and articles related to the user's health condition. For example, when a user with diabetes selects the educational materials option 502, the resources user interface may display one or more documents, videos, articles related to diagnosis, management, treatment options related to diabetes.

For example, selecting the hospital visit instructions option 504 may display one or more set of notes and documents related to the user's upcoming hospital or clinical visit. For example, the set of notes may include reminders for the user to adhere to before the user's visit, such as no food after 9 PM on the night before the hospital visit. Selecting the hospital visit instructions option 504 may also display one or more documents that the user is required to review and sign prior to their arrival for the visit.

For example, selecting the discharge instructions option 506 may display a list of instructions for the user to follow after the user is discharged from the hospital. For example, the discharge instruction may include instructions regarding the user's medications, nutrition, physical activity limitations, etc. Other types of discharge instructions are also possible.

In some examples, the data tracking module 214 of the patient care management engine 110 may track whether the user has scrolled through and otherwise complied with the hospital visit instructions and/or discharge instructions and send reminders as needed to ensure that the patient is complying with the requirements. The data tracking module 214 may also automatically document the user's progress and send communications to the user's healthcare provider and/or associated hospital or clinical administrative staff regarding the user's progress.

For example, selecting the search experts option 508 may display a search bar that the user may use to input search terms and select filters to find a healthcare provider that meets the needs of the user. For example, the user may input a health condition, location, type of expert and other variables into a search bar and receive one or more healthcare experts that meet the user's requirements. In some examples, the search feature may have one or more options to narrow down or sort the search results based on one or more criteria, experts with the greatest number of publications, experts that are located in a certain geographical location and experts affiliated with a certain hospital or institution. Other types of criteria are also possible.

In addition to a list of potential matches, the resources user interface may also recommend a particular expert from the list of potential matches. For example, the resources module 210, using machine learning models that may be trained to predict the user's selections based on the user's past preferences, similar users' preferences and ratings associated with the expert, may identify an expert that the user is likely to select and present the expert as a recommended expert to the user.

For example, selecting the search articles option 510 may display a search bar that the user may use to input search terms to find articles, research papers, and other publications related to the search term. The search article option is further described in relation to FIG. 6.

FIG. 6 illustrates an example search articles interface 600. For example, selecting the search articles option 510 on the resources user interface 500, as illustrated in FIG. 5, may cause the patient care management application 104 to display the search articles interface 600 on the display screen of the user electronic computing device 102. The search articles interface 600 is configured to receive instructions from the patient care management engine 110 to include a search bar 602. For example, the user may type in a search term into the search bar. Upon executing the search, the search articles interface 600 may display one or more search results 604. Each of the one or more search results 604 may include a link to an article, research paper, or other publication related to the search term. For example, each search result 604 may also include a title of the article and a few lines from the article so that the user may quickly preview the article. Selecting the search result 604 may display the full article, research paper or publication. Alternatively, or additionally, the user may save a copy of the article, research paper of publication associated with the search result 604 by selecting the save icon 606. Selecting the save icon 606 may result in a copy of the article, research paper of publication associated with the search result 604 being saved on the local machine or into a cloud database for the user to access in the future.

FIG. 7 illustrates an example user request user interface 700. The example user request user interface 700 may be configured to receive instructions from the patient care management engine 110 to display selectable options to one or more categories of user requests. For example, the selectable options to the one or more categories of user requests may be customized to the needs of the user. In the present example from FIG. 7, the user request user interface 700 may include a food delivery option 702, a grocery delivery option 704, a caregiver assistance option 706, a durable medical equipment option 708, a transportation option 710, a medication services option 712 and a cleaning service option 714. More or fewer number of categories of user request options are possible based on the needs of the patient. For example, user requests may also include a request related to a medical need, wherein a patient or caregiver may make a request for consultation with a medical provider or a specialist. Such user requests may be routed to the appropriate party using a virtual care management application such as the one described in U.S. patent application Ser. No. 17/655,808, titled “Remote Care Management” and filed on Mar. 22, 2022, which is hereby incorporated by reference in its entirety.

For example, selecting the food delivery option 702 may display one or more user input options where the user may input a request for food delivery from one or more restaurants, including the menu items, the delivery time, and payment options among other inputs. Based on the user's preferences set forth in the user preferences option from the list menu 308, the user's socio-economic status and user request history associated with the particular user, the user request user interface 700 may display a recommendation for a particular delivery service or in the absence of additional input from the user, may forward the user request to the particular delivery service automatically.

For example, the user may input that the user wants a burger from McDonalds and submit the request through the user request user interface 700. The user request may then be sent to the communications module 212 of the patient care management engine 110, where the user request may be processed and based on the user's preferences and socio-economic status, one of a plurality of food delivery services may be selected. For example, in the present example, Grubhub may be selected over Bite Squad or Uber Eats because the user has shown a preference for Grubhub in the past.

Once a particular entity is selected to fulfill the user request, the communications module 212 may interface with the third-party resource 114 through an API of an application associated with the entity to submit the request. Following the submission of the user request, the communications module 212 may receive confirmation information regarding when the user request will be fulfilled, the cost for the service, and other information related to the user request from the third-party resource 114. The communications module 212 may then send the confirmation information to the patient care management application 104, where the confirmation may be displayed on the user requests selectable option 318 on the main display area 302 of the patient care management user interface 300. In other examples, the patient care management application 104 may also display it as a notification or incoming message within a communications feature listed within the list menu 308.

A similar process of selecting a particular entity to process a particular user request over other entities that perform the same service and a similar process for submitting a user request may be used to fulfill all user requests, including requests related to grocery delivery, caregiver assistance, durable medical equipment delivery, transportation, medication delivery, cleaning services.

In some examples, selecting the grocery delivery option 704 may display one or more user input options where the user may input a request for grocery delivery that includes one or more grocery items. For example, the request may also indicate the particular grocery delivery service to use, or simply include a list of grocery items. Based on the user's preferences, user request history, and the user's socio-economic status, the user request user interface 700 may display a recommendation for a particular grocery delivery service or in the absence of additional input from the user, may forward the user request to the particular grocery delivery service automatically.

In some examples, selecting the caregiver assistance option 706 may display one or more user input options where the user may input a request for assistance from a caregiver. The input options may include the type of caregiver, the services the caregiver needs to perform (such as feeding the user, taking the user's vitals, etc.), an estimated budget for the caregiver service, and other information related to the caregiver. For example, the request may also indicate the particular caregiver service to use, or simply include the requirements for the caregiver. Based on the user's preferences, user request history, and the user's socio-economic status, the user request user interface 700 may display a recommendation for a particular caregiver assistance service or in the absence of additional input from the user, may forward the user request to the particular caregiver assistance service automatically.

In some examples, selecting the durable medical equipment option 708 may display one or more user input options where the user may input a request for one or more medical equipment. The input options may include the type of medical equipment, the brand name, an estimated budget range for the equipment, whether the user would like to rent or buy the equipment, and other information related to the equipment. For example, the request may also indicate the durable medical equipment service to use, or simply include the requirements for the equipment. Based on the user's preferences, user request history, and the user's socio-economic status, the user request user interface 700 may display a recommendation for a particular durable medical equipment service or in the absence of additional input from the user, may forward the user request to the particular durable medical equipment service automatically.

In some examples, selecting the transportation option 710 may display one or more user input options where the user may input a request for transportation service. The input options may include the type of vehicle (car, van), particular aspects of the vehicle (SUV, wheelchair accessible), the date and time when the service is needed, number of passengers, an estimated budget range for the transportation, and other information related to the transportation service. For example, the request may also indicate the transportation service to use, or simply include the requirements for the transportation service. Based on the user's preferences, user request history, and the user's socio-economic status, the user request user interface 700 may display a recommendation for a particular transportation service or in the absence of additional input from the user, may forward the user request to the particular transportation service automatically.

In some examples, selecting the medication services option 712 may display one or more user input options where the user may input a request for fulfillment and/or delivery of medication(s). The input options may include the type of medication (over the counter or prescription), the prescription information (for prescription medication), the user's preferences for name brand or generic medication, insurance information, an estimated budget range for the medication, whether the user would like medication to be delivered, and other information related to the medication. For example, the request may also indicate the pharmacy service to use, or simply include the requirements for the pharmacy. Based on the user's preferences, user request history, and the user's socio-economic status, the user request user interface 700 may display a recommendation for a particular pharmacy service or in the absence of additional input from the user, may forward the user request to the particular pharmacy service automatically.

In some examples, selecting the cleaning service option 714 may display one or more user input options where the user may input a request for a cleaning service. The input options may include the type of cleaning service (light housekeeping, deep cleaning, laundry, dusting, bathrooms), the number of rooms, the number of times a week the service is needed, an estimated budget range for the cleaning service, and other information related to the cleaning service. For example, the request may also indicate the cleaning service to use, or simply include the requirements for the cleaning service. Based on the user's preferences, user request history, and the user's socio-economic status, the user request user interface 700 may display a recommendation for a particular cleaning service or in the absence of additional input from the user, may forward the user request to the particular cleaning service automatically.

In some examples, in addition to displaying one or more user input options related to the selected user request, the user request user interface 700 may also display one or more recommendations or reminders for user services that the patient care management predicts the user may need in the near future. For example, based on the last medication order, the patient care management engine 110 may estimate that the user may require a refill on their medication and display a recommendation that the user should schedule a refill medication delivery. In another example, as described in greater detail in relation to FIG. 8, based on a user appointment schedule, upon selection of the transportation option 710, a message may be displayed to enquiring whether the user needs transportation services for the upcoming appointment.

FIG. 8 illustrates an example transportation request user interface 800. The transportation request user interface 800 is configured to receive instruction from the patient care management engine 110 to display a transportation service recommendation 802 for the user.

For example, the data tracking module 214 of the patient care management engine 110 may use a machine learning model to track user data to predict the user may require transportation services for an upcoming appointment. For example, the machine learning model may track user data, including the appointment times, the user's previous use of transportation services to transport the user to the appointment and the user's medical history indicating that the user is unable to currently drive a vehicle due to recent foot surgery in order to predict that the user will require a transportation service for the upcoming appointment.

In some examples, the transportation service recommendation 802 may include a predicted destination, date and time that the user may require transportation services. The transportation service recommendation 802 may also prompt the user to respond to the recommendation by selecting a response to the request 804. In the present example, the user may be presented with a plurality of responses to the request 804 for user selection, including: that the user would like to proceed with the request for transportation services to the appointment, that the user would like to request transportations services for a different time/location and that the user would not require transportation services at this time. Other types of responses are also possible.

FIG. 9 illustrates an example log patient data user interface 900. The example log patient data user interface 900 may be configured to receive instructions from the patient care management engine 110 to display selectable options to one or more categories of patient data. For example, the selectable options to the one or more categories of patient data may be customized to the needs of the user. In some instances, the user may be the patients logging in data related to their own nutrition, fluid intake, activity levels, medication intake, vitals, and other updates regarding the patient's health. In other instances, the user may be a caregiver that enters in the patient data on the patients' behalf. In addition, the caregiver may also enter check-in information to retain records regarding when the caregiver was overseeing the care of the patient.

In the present example from FIG. 9, the log patient data user interface 900 may include a nutrition log option 902, a fluid intake log option 904, an exercise log option 906, a medication log option 908, caregiver check-in log option 910, vitals log option 912, and notes option 914. More or fewer number of categories of patient data options are possible based on the needs of the patient. In some examples, the user may log data using one or more of the options 902-914 on the log patient data user interface 900. In other examples, the log patient data user interface 900 may be linked to one or more external and/or third-party peripheral devices, applications or databases such that log data related to the user may be automatically retrieved from the external or third-party devices, applications or databases. For example, the log patient data user interface 900 may automatically retrieve and store exercise data by accessing the exercise log data tracked by a smart watch. Other types of external and/or third-party devices can also be used in retrieving nutrition, fluid intake, exercise, medication, caregiver check-in, vitals information associated with the patient.

For example, selecting the nutrition log option 902 may display one or more user input options to receive data from the user regarding a patient's nutrition. For example, the user may enter one or more of the name, portion size, nutrition information of each meal or snack that the patient has consumed, and any other information related to the patient's nutrition.

For example, selecting the fluid intake log option 904 may display one or more user input options to receive data from the user regarding a patient's fluid intake. In one example, the user may enter one or more of the volume of water or other fluids consumed during the day, time of the day when the fluid was consumed and any other information related to the patient's fluid consumption. In another example, the fluid intake log option 904 may automatically retrieve data associated with the volume of fluid infused into the patient from an infusion pump device associated with the patient.

For example, selecting the exercise log option 906 may display one or more user input options to receive data from the user regarding a patient's exercise or physical activity levels. For example, the user may enter one or more of the type of activity or exercise, the duration of the activity or exercise, time of day the user performed the activity or exercise and any other information related to the patient's exercise and/or physical activity level.

For example, selecting the medication log option 908 may display one or more user input options to receive data from the user regarding a patient's medication consumption. In one example, the user may enter one or more of: the name of the medication, the amount of medication, any side effects experienced by the user, and any other information related to the patient's medication consumption. In another example, the medication log option 902 may automatically retrieve data associated with medications consumed by the patient from an infusion pump device associated with the patient.

For example, selecting the caregiver check-in log option 910 may display one or more user input options to receive data from the user regarding the caregiver's arrival and departure times. For example, the caregiver check-in log option 910 may only be available for users that are identified as caregivers. For example, the user may enter or otherwise provide through badge readers, near field communication, RFID or GPS tracking one or more of: arrival date/time, departure date/time, and any other information related to the user's time with the patient.

For example, selecting the vitals log option 912 may display one or more user input options to receive data from the user regarding the patient's vitals and health conditions. For example, the user may enter one or more of: the patient's vital information, any additional medical information related to the patient, and any other information related to the patient's vitals and health conditions. Upon receiving the patient's vial information, the data tracking module 214 may auto-document the user's vitals information.

For example, selecting the notes option 914 may display one or more user input options to receive any additional notes regarding the patient's health condition, including the patient's recovery progress.

In some examples, when the data tracking module 214 of the patient care management engine 110 receives the entered information, the data tracking module 214 may auto-document the received patient data into reports and analyze the received information to determine whether the received patient information is compliant with requirements set forth by the clinical or hospital administrators or staff members. For example, a healthcare provider may require a patient with blood pressure issues to only consume a certain amount of sodium per day. In another example, the healthcare provider may set forth the blood glucose levels for the user to maintain throughout the day. The received nutrition information may be analyzed by the data tracking module 214 to ensure that the patient is complying with the requirements. When the data tracking module 214 determines that the user has failed to comply with a requirement, the data tracking module 214 may send instructions to the patient care management application 104 to notify the user through an alert on the recent activity selectable option 324, or a message through the communications options from the list menu 308. Other ways of notifying the user are also possible.

FIG. 10 illustrates the example user engagement user interface 1000. The user engagement user interface 1000 may be configured to receive instructions from the patient care management engine 110 to display selectable options to one or more interactive games or activities. For example, the selectable interactive games options 1002 may be customized to the needs of the user. In the present example, the user engagement user interface 1000 displays a grid of nine selectable interactive games options 1002. In other examples, more or less number of selectable interactive games options 1002 may be displayed.

For example, each of the selectable interactive games options 1002 may include a name of the game or activity and a graphic related to the game or activity. Each of the selectable interactive game options 1002 may be selected for display on the user engagement user interface 1000 because the game or activity may provide the user with benefits. Each of the games may include several levels with different difficulty levels and opportunities to earn points. The games may be designed to be fun and offer rewards so that the user is motivated to continually engage with the interactive activities and games.

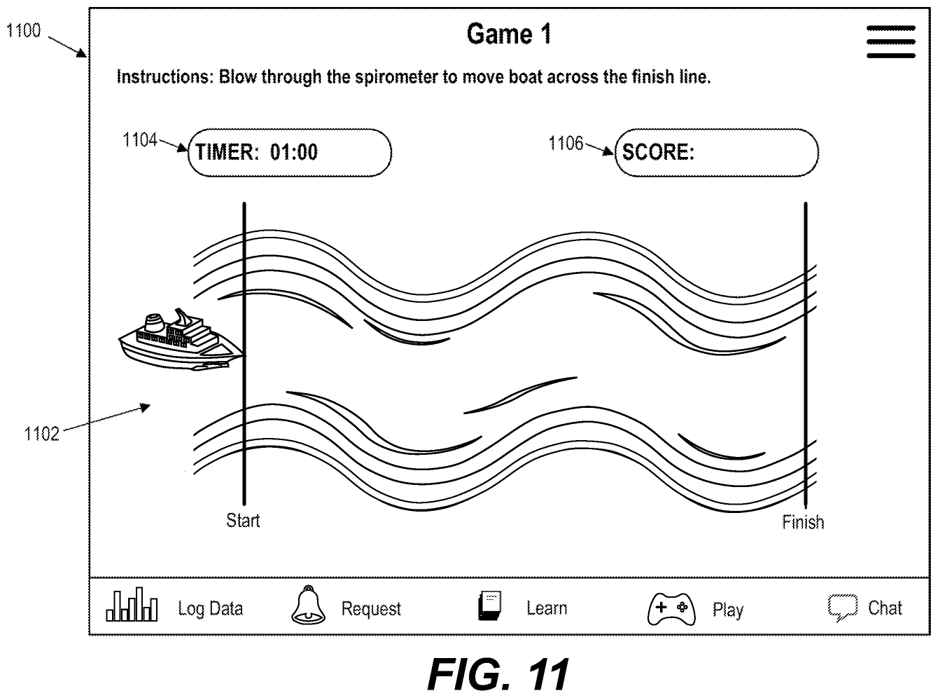

For example, the user engagement user interface 1000 for a user with diabetes may include one or more games or activities that target stabilizing the user's blood glucose levels. In another example, the user engagement user interface 1000 for a user with chronic obstructive pulmonary disease (COPD) may include one or more games that assist with improving the user's lung capacity. FIG. 11 describes one such game that helps improve lung capacity for COPD patient.

FIG. 11 illustrates an example interactive game user interface 1100. The example interactive game user interface 1100 is configured to receive instructions from the patient care management engine 110 to display an interactive game that is designed to engage a patient with COPD.

In the present example, the interactive game user interface 1100 may display a COPD game 1102 that includes a graphic of a boat on a river with start line and finish line markings. The instruction for the game may describe the object of the game. For example, the user may blow through a spirometer to move the boat across the finish line within a specific timespan to earn points.

For example, the user electronic computing device 102 may be connected to a spirometer through a wireless or wired connection such that the readings of the spirometer may be received by the user electronic computing device 102. The user may blow through the spirometer, and the measurements captured by the spirometer may be sent to the patient care management application 104 installed on the user electronic computing device 102. The amount of air the user blows through the spirometer may determine the distance the boat on the COPD game 1102 moves on the river.

The COPD game 1102 may also include a timer 1104 and a score card 1106. For example, the timer 1104 may display a clock that counts down to the provide the user with the time limit to complete the activity. The score card 1106 may display points that the user may earn by completing the game before the timer limit displayed on the timer 1104 ends. The user may also earn points by completing the game early.

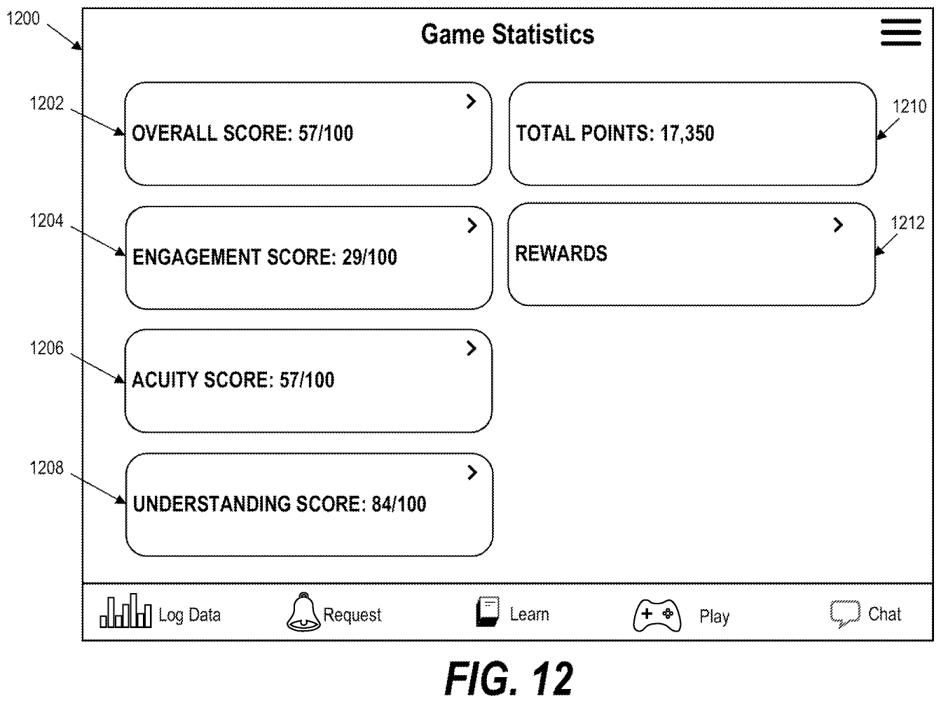

FIG. 12 illustrates an example game statistics user interface 1200. The example game statistics user interface 1200 may be configured to receive instructions from the patient care management engine 110 to display selectable options for game related data. For example, the selectable options for game related data may be based on the user's performance and engagement with the one or more games configured for the user's benefit and displayed on the user engagement user interface 1000. For example, the game statistics user interface 1200 may include one or more of: an overall score option 1202, an engagement score option 1204, an acuity score option 1206, an understanding score option 1208, a total points option 1210, and a rewards option 1212. More or fewer game statistic options may be displayed on the game statistics user interface 1200.

For example, the overall score option 1202 may display an overall score that measures the user's standing on average on the one or more games configured for the user, as described in FIG. 10. Other ways of calculating and displaying the overall score may also be possible. The overall score may also be an average score that measures the user's engagement, acuity and understanding. Selecting the overall score option 1202 may display additional information regarding the calculated overall score, including a breakdown of the overall score for different games the user has interacted with.

For example, the engagement score option 1204 may display an engagement score that measures the user's engagement with the game. For example, the engagement score may be calculated by measuring the amount of time and frequency with which the user engages with the one or more games. Other ways of calculating and displaying the engagement score may also be possible. Selecting the engagement score option 1204 may display additional information regarding the calculated engagement score, including a breakdown of the engagement score for different games that user has interacted with.

For example, the acuity score option 1206 may display an acuity score that measures the user's physical acuity. For example, the acuity score may be calculated by measuring the user's response time when the user interacts with the one or more games. Other ways of calculating and displaying the acuity score may also be possible. The acuity score may also be an average score that measures the user's acuity over a plurality of games. Selecting the acuity score option 1206 may display additional information regarding the calculated acuity score, including a breakdown of the acuity score for different games the user has interacted with.

For example, the understanding score option 1208 may display an understanding score that measures the user's understanding and mental acuity. For example, the understanding score may be calculated by measuring the user's understanding of games and the user's responses to word/puzzle games that measures the user's mental memory and acuity. Other ways of calculating and displaying the understanding score may also be possible. The understanding score may also be an average score that measures the user's understanding over a plurality of games. Selecting the understanding score option 1208 may display additional information regarding the calculated understanding score, including a breakdown of the understanding score for different games the user has interacted with.

For example, the total points option 1210 may display a total number of points that the user has acquired by interacting with the one or more games. Selecting the total points option 1210 may display additional information regarding the how the total number of points were calculated, a breakdown of total points acquired form each game the user has played, the levels achieved in each of the games, any bonuses achieved in each of the games, and other information regarding the calculated total points.

For example, selecting the rewards option 1212 may display one or more rewards that the user may qualify for based on the overall score and/or total number of points the user has earned by interacting with the one or more games available to the user. The user of a reward system may motivate the user to further engage with the interactive games or activities. For example, the user may shop for the one or more available rewards by redeeming some or all of the user's total points. The one or more rewards may be consumer products such as kitchen products, exercise products, home, decor products, clothing, shoes, jewelry, automotive products, gardening tools and prepaid gift cards. Other types of products are also possible.

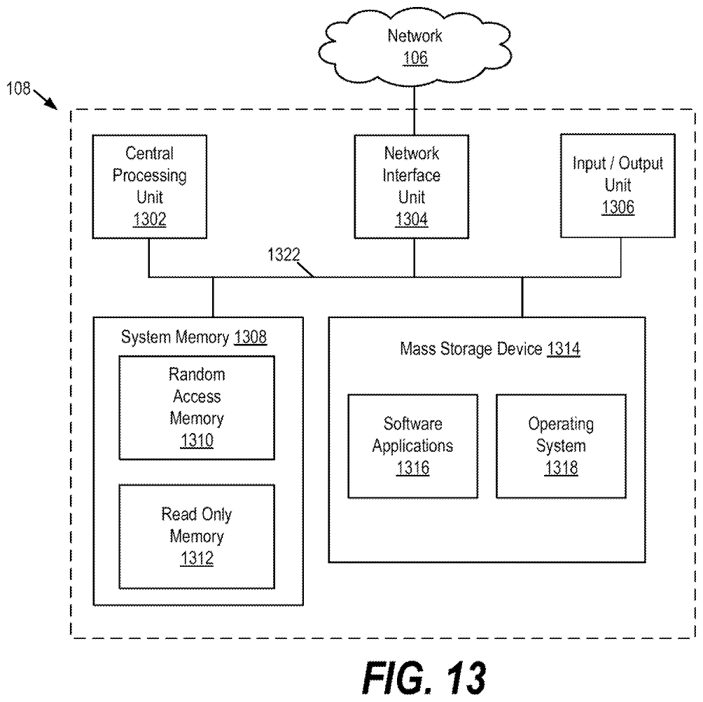

FIG. 13 illustrates example physical components of one or more of the computing devices of FIG. 1. As illustrated in the example of FIG. 13, the server computer 108 includes at least one central processing unit (“CPU”) 1302, a system memory 1308, and a system bus 1322 that couples the system memory 1308 to the CPU 1302. The system memory 1308 includes a random-access memory (“RAM”) 1310 and a read-only memory (“ROM”) 1312. A basic input/output system that contains the basic routines that help to transfer information between elements within the server computer 108, such as during startup, is stored in the ROM 1312. The server computer 108 further includes a mass storage device 1314. The mass storage device 1314 is able to store software instructions and data 1316 associated with software applications 1316. Some or all of the components of the server computer 108 can also be included in user electronic computing device 102.

The mass storage device 1314 is connected to the CPU 1302 through a mass storage controller (not shown) connected to the system bus 1322. The mass storage device 1314 and its associated computer-readable data storage media provide non-volatile, non-transitory storage for the server computer 108. Although the description of computer-readable data storage media contained herein refers to a mass storage device, such as a hard disk or solid-state disk, it should be appreciated by those skilled in the art that computer-readable data storage media can be any available non-transitory, physical device or article of manufacture from which the central processing unit can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the server computer 108.

According to various embodiments of the invention, the server computer 108 may operate in a networked environment using logical connections to remote network devices through the network 106, such as a wireless network, the Internet, or another type of network. The server computer 108 may connect to the network 106 through a network interface unit 1304 connected to the system bus 1322. It should be appreciated that the network interface unit 1304 may also be utilized to connect to other types of networks and remote computing systems. The server computer 108 also includes an input/output controller 1306 for receiving and processing input from a number of other devices, including a touch user interface display screen, or another type of input device. Similarly, the input/output controller 1306 may provide output to a touch user interface display screen or other type of output device.

As mentioned briefly above, the mass storage device 1314 and the RAM 1310 of the server computer 108 can store software instructions and data associated with software applications 1316. The software instructions include an operating system 1318 suitable for controlling the operation of the server computer 108. The mass storage device 1314 and/or the RAM 1310 also store software instructions, that when executed by the CPU 1302, cause the server computer 108 to provide the functionality of the server computer 108 discussed in this document. For example, the mass storage device 1314 and/or the RAM 1310 can store software instructions that, when executed by the CPU 1302, cause the server computer 108 to display received data on the display screen of the server computer 108.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A method for providing patient care, the method comprising:
delivering pre-hospital information to a patient in a graphical user interface, the pre-hospital information including information about treatments for the patient and a pre-operation virtual consult with a caregiver; and
delivering post-hospital information to the patient in the graphical user interface, the post-hospital information including information about medications and rehabilitation for the patient.

2. The method of claim 1, further comprising:
based on a discharge summary associated with the patient, automatically scheduling one or more appointments for the patient.

3. The method of claim 1, further comprising:
receiving information related to one or more of: nutrition, fluid intake, vitals, and activity levels of the patient; and
automatically documenting the information.

4. The method of claim 1, wherein, the information related to the patient is received from a user electronic computing device.

5. The method of claim 1, wherein the pre-hospital information further includes one or more consent forms associated with patient's upcoming hospitalization.

6. The method of claim 1, further including:
tracking whether the patient has reviewed the pre-hospital information;
based on the tracking, generating a compliance report describing whether the patient has complied with pre-hospitalization requirements; and
sending the compliance report to the caregiver.

7. A method for providing patient care outside of a healthcare facility, the method comprising:
identifying needs of a patient located outside of the healthcare facility;
routing one or more of the needs to a caregiver of the patient;
providing a dashboard for the patient, the dashboard including:
capturing patient information from the patient and the caregiver; and
accessing the patient information for the caregiver.

8. The method of claim 7, wherein the patient is located in a residence of the patient.

9. The method of claim 7, wherein the caregiver is a family member.

10. The method of claim 7, wherein the patient information is one or more of: nutrition, fluid intake, vital signs and activity levels of the patient.

11. The method of claim 7, further comprising:
capturing caregiver information from the caregiver, wherein the caregiver information includes arrival time and departure time.

12. The method of claim 11, wherein the caregiver information is captured by scanning a badge or radio-frequency identification (RFID) tag associated with the caregiver or tracking a location of the caregiver using a real-time location system (RTLS).

13. The method of claim 7, wherein the dashboard further includes:
routing at least one of the needs to a third-party service.

14. The method of claim 13, wherein the third-party service is one of: a food delivery service, a grocery delivery service, a pharmacy service, a cleaning service, or a transportation service.

15. The method of claim 7, wherein the needs of the patient are identified by analyzing the patient information.

16. A method of gamification of a patient care, the method comprising:
identifying a health condition of a patient;
identifying an electronic game that aids in improving the health condition; and
providing the electronic game on a user electronic computing device of the patient, wherein the patient engages with the electronic game through the user electronic computing device.

17. The method of claim 16, wherein the patient further engages with the electronic game through one or more medical devices.

18. The method of claim 16, further comprising:
calculating a score based on a patient engagement level, wherein the patient engagement level is measured based on a frequency and an amount of time the patient interacts with the electronic game.

19. The method of claim 18, wherein the patient earns rewards based on the score.

20. The method of claim 19, further comprising:
delivering pre-hospital information to the patient in a graphical user interface through the user electronic computing device, the pre-hospital information including information about treatments for the patient and a pre-operation virtual consult with a caregiver;
delivering post-hospital information to the patient in the graphical user interface through the user electronic computing device, the post-hospital information including information about medications and rehabilitation to treat the health condition of the patient.
